(19) United States
(12) Patent Application Publication
(10) Pub. No.: US 2007/0162127 A1
(43) Pub. Date: Jul. 12, 2007

(54) Instruments and techniques for delivering non-rigid implant members in surgical procedures

(75) Inventors: Marc M. Peterman, Austin, TX (US); Kent Anderson, Memphis, TN (US)

Correspondence Address: KRIEG DEVAULT LLP, ONE INDIANA SQUARE, SUITE 2800, INDIANAPOLIS, IN, 46204-2709, US

(73) Assignee: SDGI Holdings, Inc.

(21) Appl. No.: 11/297,732

(22) Filed: Dec. 8, 2005

(51) Int. Cl. A61F 2/44

(52) U.S. Cl. 623/17.11

(57) ABSTRACT

Instruments and techniques for implanting non-rigid implants are provided that include positively engaging the implant between distal and proximal ends of the implant at least adjacent the distal end of the insertion instrument while deploying the implant longitudinally and distally from the insertion instrument.

### BACKGROUND

The present invention relates to prosthetic device implantation, and more particularly, but not exclusively, relates to implants and to instruments and techniques for delivering non-rigid implant members to a location in a patient in a surgical procedure.

The use of prosthetic implants to address orthopedic injuries and ailments has become commonplace. Nonetheless, there is an ever-present challenge to enable less invasive surgical techniques, improve implant delivery capabilities to the surgical site, shorten the time required to surgically implant prosthetic devices, decrease surgery recovery time, and/or provide other improvements. Thus, additional contributions in this area of technology remain desirable.

### SUMMARY

One embodiment of the present application is a unique spinal implantation technique. Other embodiments include unique methods, systems, devices, kits, tools, instrumentation, and apparatus involving implantation of a non-rigid prosthetic device within a patient.

In one aspect, there is provided a surgical method that includes mounting an implant along a distal end portion of an insertion instrument, the implant including a non-rigid, elongated body extending between distal and proximal ends; positioning at least the distal end portion of the insertion instrument adjacent at least one spinal element, the insertion instrument extending along a longitudinal axis; and deploying the implant longitudinally and distally from the distal end portion of the insertion instrument with a deployment force delivered along the longitudinal axis at the distal end portion of the insertion instrument and applied to the implant between the distal and proximal ends of the implant.

In another aspect, there is provided a system that includes an implant with an elongated, non-rigid body extending between a distal end and a proximal end. The system also includes an insertion instrument extending along a longitudinal axis. The implant can be mounted along a distal end portion of the insertion instrument. The insertion instrument includes an elongated deployment member extending along and axially moveable relative to an elongated retaining member that is contactable with the implant. The deployment member includes a distal engaging portion engaging the implant. Relative axial movement between the deployment member and the retaining member is operable to deploy the implant longitudinally and distally from the distal end of said insertion instrument while the engaging portion engages the implant between the proximal and distal ends of the implant.

In a further aspect, an insertion instrument for inserting a non-rigid implant includes a deployment member engageable with the implant and an engaging portion to deliver a deployment force to the implant adjacent a distally end of the insertion instrument. A retaining member is received in a passage of the deployment member. The deployment member and the retaining member are movable proximally and sequentially relative to one another to deploy the implant distally and axially from the distal end of the insertion instrument.

In another aspect, an insertion instrument includes a deployment member, a retaining member in the deployment member, an engaging portion for engaging the implant, a retaining member, and a compression member engageable with the distal end of the implant. The deployment member, retaining member and compression member are operable to deploy the implant distally and longitudinally of the insertion instrument in an inch-worm like manner.

In another aspect, an insertion instrument for inserting a non-rigid implant includes a retaining member for mounting the implant to the insertion instrument and a belt movable along the retaining member to deploy the implant distally and axially from the insertion instrument.

Further embodiments, forms, features, aspects, benefits, objects, and advantages of the present application shall become apparent from the detailed description and figures provided herewith.

### BRIEF DESCRIPTION OF THE DRAWINGS

FIG. 1 is a partial perspective view of a distal portion of an implant insertion instrument.

FIGS. 2A and 2B are an elevation view and a partial section view, respectively, of the instrument in FIG. 1 with an implant positioned thereon for delivery to a surgical site.

FIGS. 3A and 3B are an elevation view and a partial section view, respectively, of the instrument and implant with the deployment member of the instrument proximally retracted.

FIGS. 4A and 4B are an elevation view and a partial section view, respectively, of the instrument and implant with the retaining member of the instrument proximally retracted relative to its positioning in FIGS. 3A and 3B to initially deploy the implant.

FIGS. 5A and 5B are an elevation view and a partial section view, respectively, of the instrument and implant with a portion of the implant finally deployed distally of the instrument for implantation at the surgical site.

FIGS. 6A and 6B are elevation view of a portion of another embodiment implant in a deployed configuration and an implantation configuration, respectively.

FIGS. 7A and 7B are elevation view of a portion of another embodiment implant in a deployed configuration and an implantation configuration, respectively.

FIGS. 8A and 8B are sectional views the portion of the implant of FIGS. 7A and 7B, respectively.

FIG. 9 is a perspective view of a distal portion of another embodiment of the insertion instrument of FIG. 1.

FIG. 10 is a longitudinal sectional view of the instrument of FIG. 9 with an implant mounted thereto.

FIG. 11 is a perspective view of another embodiment insertion instrument.

FIG. 12 is a longitudinal sectional view of a distal portion of the insertion instrument of FIG. 11 with an implant positioned therein.

FIG. 13 is a longitudinal sectional view of the distal portion of the insertion instrument with of FIG. 11 with the implant being delivered distally therefrom for implantation at the surgical location.

FIG. 14 is a longitudinal sectional view of a distal portion of another embodiment of the insertion instrument of FIG. 11.

FIG. 15 is a diagrammatic view illustrating examples of implanted arrangements for implants delivered with the insertion instruments herein.

FIG. 16 is a diagrammatic view illustrating further examples of implanted arrangements for implants delivered with the insertion instruments herein.

### DETAILED DESCRIPTION OF SELECTED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

Non-rigid implants for implantation in the human body can be desired for tissue repair, to provide scaffolds between adjacent anatomical elements for structural support and tissue growth, and for other beneficial reasons. Delivery of such implants in a desired configuration and/or at a desired location in the patient's body can be difficult since the implants can alter in form or shape if a trailing end of the implant is pushed during implantation and movement of the implant encounters resistance. Instruments and techniques for implanting non-rigid implants are provided that include positively engaging the implant between distal and proximal ends of the implant at least adjacent the distal end of the insertion instrument while displacing the implant longitudinally and distally from the insertion instrument with the deployed portion of the implant unsupported by the insertion instrument.

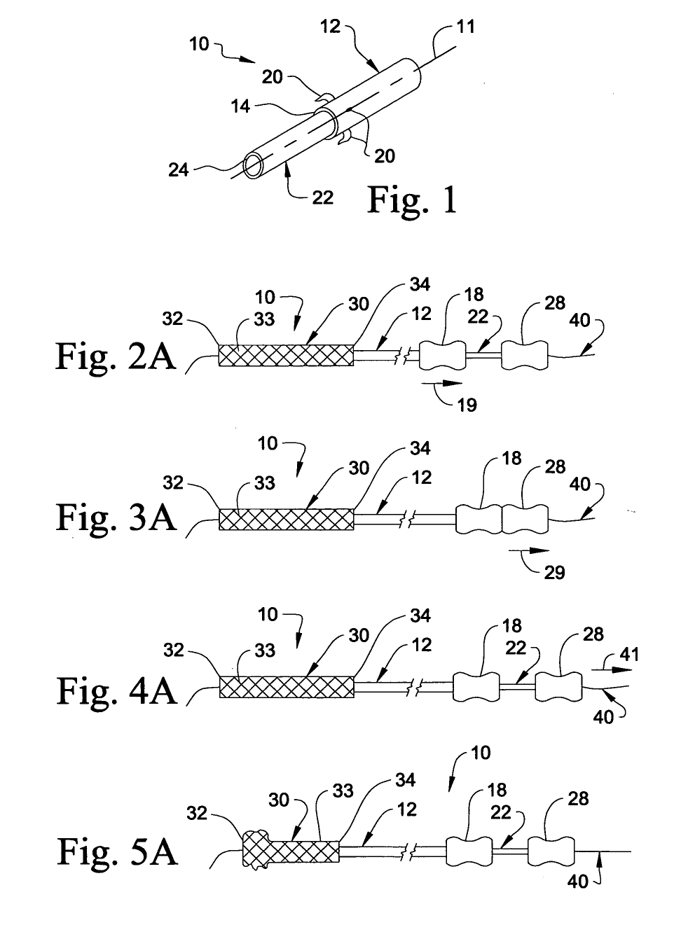

FIG. 1 illustrates a distal portion of one embodiment insertion instrument 10 for delivering and deploying a non-rigid spinal implant to a location in a patient's body in a surgical procedure. In the illustrated embodiment, instrument 10 includes a deployment member 12 and a retaining member 22. Deployment member 12 can be in the form of an elongated tube having a lumen for receiving retaining member 22 therethrough. Retaining member 22 can be in the form of an elongated tube having a lumen extending therethrough. Deployment member 12 and retaining member 22 can be moved longitudinally relative to one another along longitudinal axis 11 to deploy the implant at the implantation location.

In the illustrated embodiment, deployment member 12 includes at least one engaging portion 20, the purposes for which will be discussed further below. The at least one engaging portion 20 can further include a plurality of hook-shaped engaging portions with distally oriented barbs or tines spaced about the outer surface of deployment member 12. The proximally oriented sides of engaging portions 20 can be smooth to facilitate proximal displacement of deployment member 12 within implant 30.

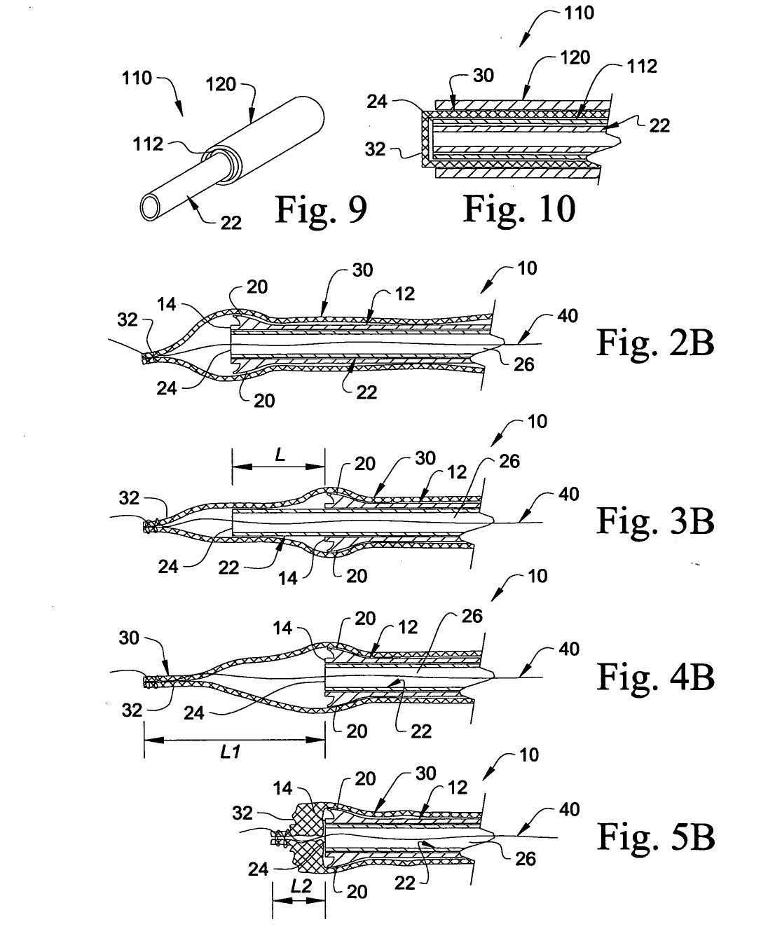

Referring now to FIGS. 2A and 2B, instrument 10 is shown with the distal end 24 of retaining member 22 aligned longitudinally with distal end 14 of deployment member 12. An implant 30 is shown mounted about deployment member 12 and retaining member 22. Implant 30 includes a distal end 32 located distally of the distal ends of deployment member 12 and retaining member 22. Implant 30 can be tubular in form to define a central bore and allow it to be positioned about instrument 10. Implant 30 can further include a non-rigid body 33 so that it can be collapsed, compressed, expanded, and/or conformable in response to external forces that are applied to it.

Distal end 32 of implant 30 can be closed or blocked, either completely or partially, by securing the walls of implant 30 together with a suture, adhesive, bonding, connector, or fastener, or by providing implant 30 with an end cap, end wall, end flange, or other suitable arrangement. An axial compression member 40 is engaged to distal end 32 and extends therefrom through lumen 26 of retaining member 22. In the illustrated embodiment of FIG. 2A, the tubular configuration of implant 30 is carried through to its distal end 32. Compression member 40 can be engaged to the distal end of implant 30. An alternative configuration for implant 30 in FIG. 2B shows distal end 32 of implant 30 collapsed to a closed or substantially closed configuration and maintained as such with compression member 40.

The proximal end of deployment member 12 can include a handle portion 18, and the proximal end of retaining member 22 can include a handle portion 28. Handle portions 18, 28 can be manipulated to move deployment member 12 and retaining member 22 relative to one another to axially and longitudinally deploy implant 30 from the distal ends thereof. In the illustrated embodiment, handle portions 18, 28 are enlarged to facilitate manual gripping of deployment member 12 and retaining member 22 and in the application of longitudinally directed forces to move members 12, 22 relative to one another.

Other handle configurations are also contemplated. For example, a pistol grip could be provided at the proximal end, with triggers coupled to respective ones of the members 12, 22 to allow manual manipulation of the members 12, 22 relative to one another. Compression member 40 can extend proximally through the pistol grip to a knob, ratcheting mechanism or other device or tool that facilitates proximal displacement of compression member 40 and maintenance of the positioning thereof relative to members 12, 22.

In the initial configuration of FIGS. 2A and 2B, insertion instrument 10 and implant 30 can be positioned into the patient so that implant 30 is located at the surgical site. Deployment member 12 and retaining member 22 can be elongated so implant 30 can be delivered with insertion instrument 10 to locations deep within the patient's body, and/or through minimally invasive access portals formed by any one or combination of micro-incisions, sleeves, cannulas, and retractors, for example. Deployment member 12 and retaining member 22 can also be flexible to facilitate positioning around anatomical structures and surgical instruments that may obstruct access to the surgical site. Embodiments where one or both of members 12, 22 are rigid are also contemplated.

In FIGS. 2A and 2B, instrument 10 includes a first configuration where handle portion 18 is spaced distally of handle portion 28, and distal ends 14, 24 of members 12, 22 are aligned with or generally adjacent to one another. Handle portion 18 can then be displaced proximally as indicated by arrow 19 to its positioning in FIGS. 3A and 3B. In this second configuration, deployment member 12 is displaced longitudinally and proximally about retaining member 22 so that distal end 14 is spaced proximally of distal end 24 of retaining member 22 by a distance L. Distance L corresponds to an initial deployment length for implant 30. Contact between distal end 32 of implant 30 and distal end 24 of retaining member 22 maintains implant 30 in longitudinal position as deployment member 12 is retracted proximally. Retaining member 22 maintains the implant shape as it is received in the internal bore of implant 30.

From the second configuration, handle 28 can be displaced proximally as indicate by arrow 29 to a third configuration for instrument 10 shown in FIGS. 4A and 4B. In the third configuration, retaining member 22 is moved longitudinally and proximally relative to deployment member 12 while the longitudinal positioning of implant 30 is maintained to deploy implant 30 distally and longitudinally of insertion instrument 10. Retaining member 22 is moved until distal ends 14, 24 are aligned with or generally adjacent to one another, providing implant 30 with an unsupported length extending distally from distal ends 14, 24 that corresponds to deployment length L1. Retaining member 22 can be provided with indicia or other markings therealong to provide an indication of the deployment length L1 for implant 30.

As retaining member 22 is moved proximally, implant 30 may tend to follow retaining member 22 proximally and slide proximally along deployment member 12 due to frictional engagement of implant 30 with retaining member 22. Deployment member 12 engages the inner wall of implant 30 between its distal and proximal ends to apply a deployment force that maintains implant 30 in position longitudinally and distally of the distal end of members 12, 22 as retaining member 22 is displaced. In the illustrated embodiment, this deployment force is provided by engaging members 20 adjacent the distal end 14 of deployment member 12 that prevent implant 30 from bunching or sliding proximally along deployment member 12.

In FIGS. 5A and 5B, compression member 40 is tensioned proximally as indicated by arrow 41 in FIG. 4A. Compression member 40 displaces distal end 32 of implant 30 proximally along longitudinal axis 11 toward insertion instrument 10. During this proximal displacement, deployment member 12 maintains engagement with implant 30 to prevent it from bunching along instrument 10 or from sliding proximally along instrument 10. This in turn causes implant 30 to compress against distal ends 14, 24 to form a finally deployed implanted portion having an implantation length L2 along axis 11 that is less than the deployment length L1. In the illustrated embodiment, implant 30 bunches when compressed and thus radially expands to provide implant 30 with a height greater than when in the initially deployed configuration. Other embodiments contemplate that implant 30 can be structured to fold in an accordion-like manner, or that the distal end of implant 30 is pulled through its inner bore.

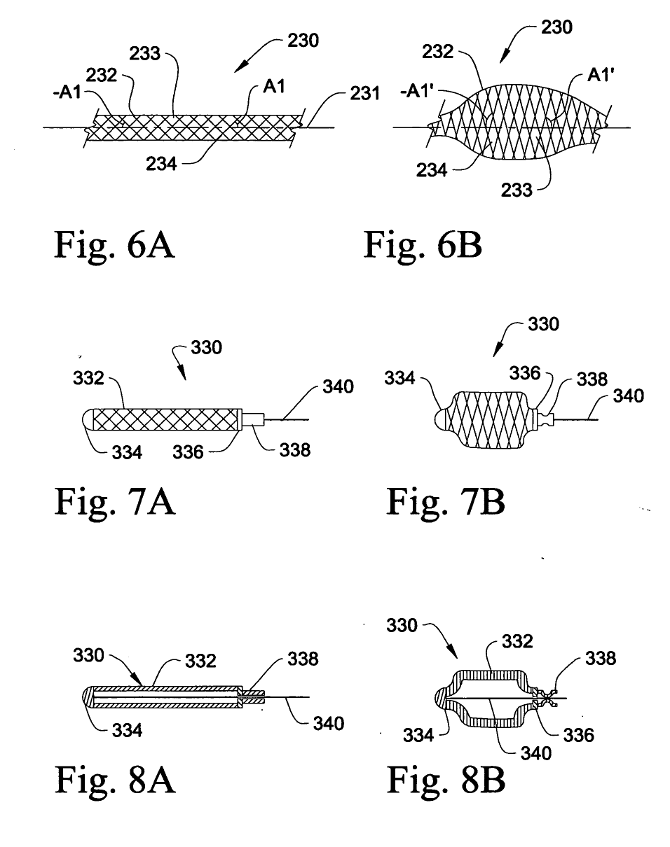

Another embodiment implant 230 is shown in FIGS. 6A and 6B. Implant 230 can be mounted to instrument 10 and deployed from the same in a manner similar to implant 30. Implant 230 includes a body 232 forming a hollow braided structure formed by first transverse components 233 and second transverse components 234. First and second components 233, 234 are transversely oriented to one another, and each form an acute angle with longitudinal axis 231. In the relaxed or tensioned configuration shown in FIG. 6A, the angular orientation of with components 233, 234 with respect to axis 231 is A1, −A1, respectively.

When the distal end 236 of body 232 is displaced axially, as shown in FIG. 6B, components 233, 234 re-orient relative to longitudinal axis 231 to angles A1′, −A1′, respectively. This re-orientation of components occurs due to shortening of the deployed length of implant 230 shown in FIG. 6A to its implanted length shown in FIG. 6B. The angles A1′, −A1′ formed by the compressed length of implant 230 are greater than the angles A1, −A1 formed by the uncompressed length of implant 230. Compression of body 232 can change a pliable body structure into a shorter, stiffer implant suitable for supporting compression loads from the spinal column while maintaining spacing between vertebrae.

Another embodiment implant is shown in FIGS. 7A-8B. Implant 330 includes a body 332 extending between a distal end cap or member 334 and a proximal end member 336. When compression member 340 is tensioned to compress implant 330 from the relaxed or tensioned state of FIGS. 7A, 8A to the compressed state of FIGS. 7B, 8B, the length of implant 330 is reduced and body 332 radially expands. A crimp 338 can be engaged about compression member 340 adjacent to proximal end member 336, maintaining implant 330 in its implanted configuration. Crimp 338 maintains body 332 in a compressed state between end members 334, 336 when subjected to compression loads from the spinal column.

These steps for deploying portions of the length of the implants 30, 230, 330 are repeated until the implant is totally deployed at the implantation location. When implant 30, 230, 330 is deployed, compression member 40 can be secured against the proximal end of the implant to maintain the implant in its implanted configuration. In one form, compression member 40 is a suture and a knot is provided therein for securement against the proximal end of implant 30, 230, 330. Compression member 40 can thus extend through implant 30 for securement against the opposite ends thereof to maintain the implant in its deployed condition. In other forms, an engagement member such as an end cap, anchor or other device is delivered along compression member 40 to secure and maintain the implant in its deployed configuration.

The finally deployed height of implant 30, 230, 330 transverse to longitudinal axis 11 can be selected to occupy a space in the implantation location between adjacent anatomical structures. Implant 30, 230, 330 can be flexible and compressible to conform to the size and shape of the space in which it is implanted. In another form, implant 30, 230, 330 in the implanted configuration can act on one or more anatomical elements when deployed to provide a desired configuration, spacing or other relationship to the anatomical element or between the anatomical elements.

Referring now to FIGS. 9 and 10, there is shown another embodiment insertion instrument 110. Insertion instrument 110 can be configured similarly to insertion instrument 10 discussed above, and includes a deployment member 112 positioned about retaining member 22. Implant 30 can be positioned about deployment member 112 for delivery and deployment at the surgical location. Insertion instrument 110 differs from instrument 10 discussed above in that rather than engaging members on the outer surface of deployment member 112, an engaging member 120 is provided in the form of a cannula positioned about deployment member 112. Implant 30 is received between engaging member 120 and deployment member 112. Deployment member 112 and engaging member 120 engage implant 30 to provide a deployment force between the proximal and distal ends of implant 30 at the distal end of insertion instrument 110. The deployment force prevents gathering of implant 30 and/or sliding or implant 30 proximally along deployment member 112 during proximal displacement of retaining member 22 into deployment member 112 when initially deploying implant 30.

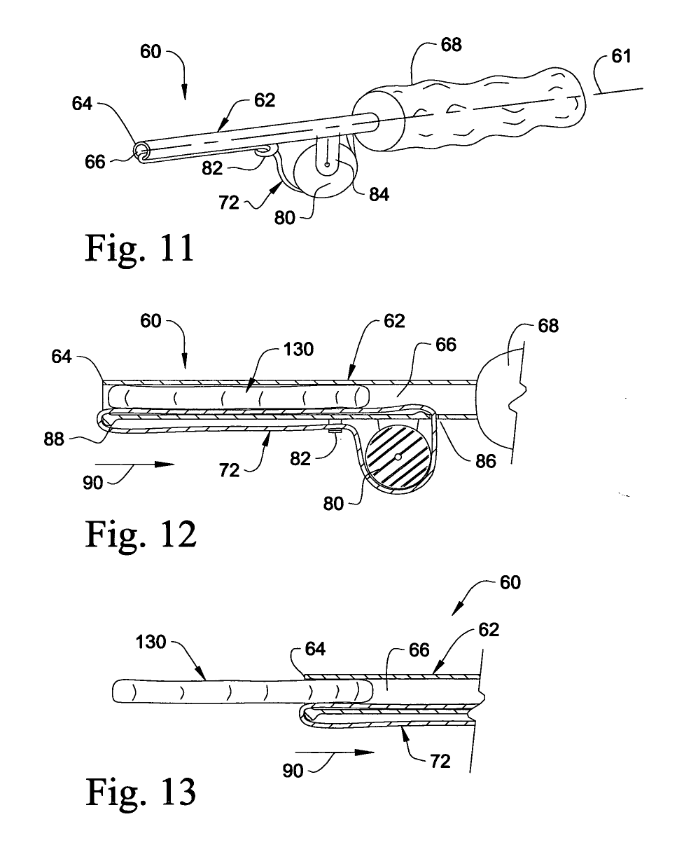

Referring now to FIGS. 11-13, there is shown another embodiment insertion instrument for delivering non-rigid implants to an implantation location within a patient. Insertion instrument 60 includes an elongated retaining member 62 extending along a longitudinal axis 61 and a deployment member 72 mounted to retaining member 62. Retaining member 62 includes a distal end 64 opening into an elongated passage 66 defined by retaining member 62. A handle portion 68 is mounted to the proximal end of retaining member 62. In the illustrated embodiment, retaining member 62 is a tube having a wall that completely encloses passage 66 along a majority of the length of retaining member 62.

Various forms for the cross-sectional shape of retaining member 62 and passage 66 are contemplated, including circular shapes, C-shapes, U-shapes, D-shapes, rectangular shapes, V-shapes, oval shapes, polygonal shapes, and irregular shapes, for example. Retaining member 62 can also be adapted with multiple passages to deliver multiple implants to the surgical location, or to provide one or more access pathways to the implantation location for delivery of biomaterials, other implants, viewing instruments, surgical instruments, and therapeutic substances, for example. Retaining member 62 can be rigid, flexible, or include flexible portions to assist in maneuvering insertion instrument 60 around anatomical structures, surgical instruments, and other obstructions to accessing the implantation location.

Implant 130 is deliverable to an implantation location with insertion instrument 60. Implant 130 can include an elongated body sized to be received in insertion instrument 60 and structured to permit delivery thereof with insertion instrument 60. The body of implant 130 can be non-rigid and can also be solid, although the use of bodies with one or more bores extending therealong is also contemplated.

Deployment member 72 is received within passage 66 and is moveable therealong to deploy implant 130 positioned in passage 66 through the distal end opening of retaining member 62. In the illustrated embodiment, deployment member 72 is an elongated belt-like member that forms a continuous loop. A mounting member 80 is provided on retaining member 62 adjacent its proximal end, and provides a wheel about which at least a portion of deployment member 72 extends.

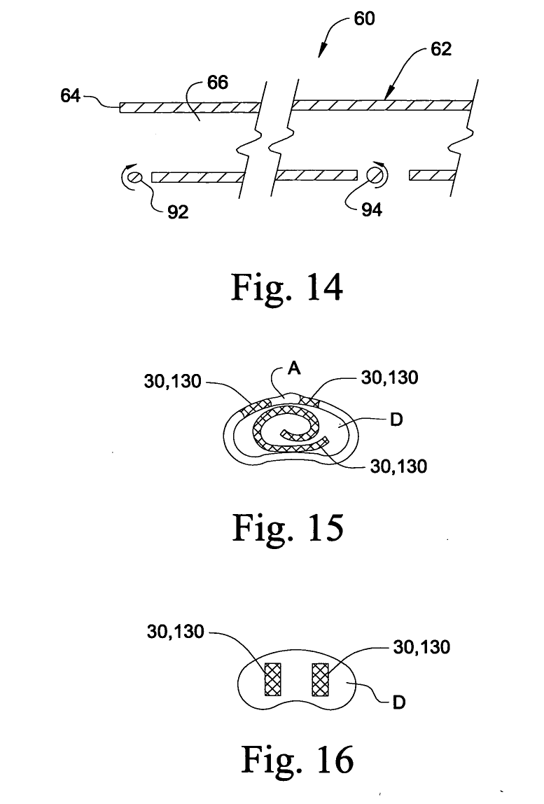

Deployment member 72 enters passage 66 at a penetration 86 extending through the wall of retaining member 62 at a location adjacent to mounting member 80. Deployment member 72 extends along passage 66 to distal end 64 of retaining member 62. Deployment member 72 further extends about distal end 64 and then proximally toward mounting member 80. Distal end 64 can include a rounded lip 88 to provide a smooth, less abrupt transition about which deployment member 72 extends and to facilitate sliding movement of deployment member 72 about distal end 64. In another embodiment shown in FIG. 14, retaining member 62 includes a distal roller 92 and a proximal roller 94 to facilitate movement of deployment member relative thereto. A tensioner 82 extends from retaining member 62 adjacent to and distally of mounting member 80. Deployment member 72 extends through tensioner 72, which facilitates maintenance of tension on deployment member 72 and to maintain deployment member 72 in a low profile arrangement relative to retaining member 62.

As shown in FIG. 12, an elongated implant 130 can be positioned in passage 66 along deployment member 72. Mounting member 80 can be manipulated with the thumb, fingers or instrument to rotate deployment member 72 in a clockwise direction, as indicated by arrow 90. The portion of deployment member 72 within passage 66 is displaced distally while frictionally engaging implant 130 along a substantial portion of its length in passage 66. Accordingly, as deployment member 72 is rotated in the direction of arrow 90, implant 130 is displaced distally in passage 66 and delivered through the distal end opening of retaining member 62.

Deployment member 72 contacts implant 130 along the length of implant 130 within passage 66, and the delivery force is therefore distributed along this supported portion of the length of implant 130. Deployment member 72 allows a delivery or deployment force to be applied to the implant at the distal end of the implant carrier, i.e. retaining member 62, to a location proximal of the distal end of retaining member 62. In the illustrated embodiment, deployment member 72 has a length at least as great as the length of the implant. Other embodiments contemplate the implant is provided with a length that is greater than the length of deployment member 72. The arrangement of deployment member 72 relative to retaining member 62 prevents implant 130 from radially expanding or bunching within passage 66 as it is passed through the distal end opening of retaining member 62, facilitating smooth and rapid delivery of implant 130 through retaining member 62 to the implantation location.

The implants discussed herein can be employed in a variety of surgical procedures. One contemplates an annulus repair technique where at least one implant is placed in or adjacent a defect formed in a wall of annulus A extending about a spinal disc space, such as shown in FIG. 15. The implant can be positioned to extend transversely to the annulus, or to extend along the annulus perimeter. The at least one implant can block all or a portion of the defect or void within the annulus fibrosis, such as may be caused by surgery or disc herniation. The at least one implant can be engaged to soft tissue and/or hard tissue or bone adjacent to the defect or void with anchors, sutures, spikes or any other suitable engagement structures to assist in retaining implants 30, 130 in a substantially fixed position relative to the defect or void relative to adjacent soft or hard tissue.

Another technique contemplates one or more implants positioned within a spinal disc space D as shown in FIGS. 15 and 16, for example. The implants can be positioned along or in combination with other implants in the spinal disc space. One or more implants can be positioned to extend linearly, as shown in FIG. 16, or non-linearly, as shown in FIG. 15. The implants can be flexible and function as a nucleus replacement to allow motion between adjacent vertebrae to be maintained post-operatively. Intervertebrally positioned implants can function as a scaffold to maintain a separation or distraction spacing between vertebrae for ensuing fusion of the spinal motion segment. Intravertebrally positioned implants can function as a scaffold or reduction device to restore a fractured or deformed vertebral body. The implants and/or the space around the implant can include bone growth promoting material to facilitate bone growth and fusion between the adjacent vertebrae. Other procedures contemplate that implants 30, 130 are positioned between posterior spinal elements. In spinal surgical procedures, the implant can be delivered to one or more vertebrae or to a disc space between vertebrae in an anterior approach, a posterior approach, a lateral approach, postero-lateral approach, a transforaminal approach, an anterior oblique approach, for example.

With respect to the various implant embodiments described herein, the deployment member can be joined or fixed to the implant using various devices and/or techniques, or can be integrally formed with or an extension of the implant that is severed from the implant when it is delivered to the implantation location. The deployment member can be joined or attached to the implant by, for example, suturing or sewing, threading, thermal welding or bonding, adhesive bonding, three dimensional weaving or braiding, screws, staples, pins, tacks or rivet fixation, or frictional engagement.

The implants can be fabricated from one or more components that are flexible or exhibit at least some flexibility and are non-rigid. Some examples include extruded components, machined components, molded components, formed components, and milled components. Other examples of implant components include woven fabric tubing, woven and non-woven mesh, or braided or woven structures. The implant body can include any one or more of sheets, tethers, cords, planar members, bands, wires, cables, or any other component capable of forming or being formed into the implant body. In a further form, the implant may be resilient and/or elastic so it can assume various shapes during and after insertion and attachment while exhibiting a tendency to return to its natural form. In yet another form, the implant is substantially inelastic so that the shape achieved upon deployment is readily maintained.

Growth factors or cells can be infused, carried or otherwise incorporated into, within, and/or about the implant to accelerate the tissue growth. Growth factors can be transforming growth factor beta. 1, insulin-like growth factor 1, platelet-derived growth factor, fibroblast growth factor, bone morphogenetic protein (BMP), LIM mineralization protein (LMP) and combinations thereof Other therapeutic substances and growth stimulating substances are also contemplated.

The implants can be made from any biocompatible material, material of synthetic or natural origin, and material of a resorbable or non-resorbable nature. Suitable examples of implant material include autograft, allograft or xenograft; tissue materials including soft tissues, connective tissues, demineralized bone matrix and combinations thereof; resorbable materials including polylactide, polyglycolide, tyrosinederived polycarbonate, polyanhydride, polyorthoester, polyphosphazene, calcium phosphate, hydroxyapatite, bioactive glass, collagen, albumin, fibrinogen and combinations thereof; and non-resorbable materials including polyethylene, polyester, polyvinyl alcohol, polyacrylonitrile, polyamide, polytetrafluorethylene, polyparaphenylene terephthalamide, cellulose, carbon-reinforced polymer composites, shape memory alloys, titanium, titanium alloys, cobalt chrome alloys, stainless steel, and combinations thereof.

While the invention has been illustrated and described in detail in the drawings and foregoing description, the same is to be considered illustrative and not restrictive in character, it being understood that only selected embodiments have been shown and described and that all changes, equivalents, and modifications that come within the scope of the inventions described herein or defined by the following claims are desired to be protected. Any experiments, experimental examples, or experimental results provided herein are intended to be illustrative of the present invention and should not be construed to limit or restrict the invention scope. Further, any theory, mechanism of operation, proof, or finding stated herein is meant to further enhance understanding of the present invention and is not intended to limit the present invention in any way to such theory, mechanism of operation, proof, or finding. In reading the claims, words such as “a”, “an”, “at least on”, and “at least a portion” are not intended to limit the claims to only one item unless specifically stated to the contrary. Further, when the language “at least a portion” and/or “a portion” is used, the claims may include a portion and/or the entire item unless specifically stated to the contrary.

What is claimed is:

1. A spinal surgical method, comprising:
mounting an implant along a distal end portion of an insertion instrument, the implant including a non-rigid, elongated body extending between distal and proximal ends;
positioning at least the distal end portion of the insertion instrument adjacent at least one spinal element, the insertion instrument extending along a longitudinal axis; and
deploying the implant longitudinally and distally from the distal end portion of the insertion instrument with a deployment force delivered along the longitudinal axis at the distal end portion of the insertion instrument and applied to the implant between the distal and proximal ends of the implant.

2. The method of claim 1, wherein mounting the implant includes mounting the implant about a distal end of an elongated deployment member of the insertion instrument, the deployment member including an implant retaining member axially movable therein.

3. The method of claim 2, wherein deploying the implant includes the steps of:
a) proximally displacing the deployment member relative to the retaining member and the implant while maintaining axial positioning of the implant with the retaining member, wherein the retaining member includes a distal end portion supporting the implant during proximal displacement of the deployment member.

4. The method of claim 3, wherein deploying the implant includes the steps of:
b) proximally displacing the retaining member relative to the deployment member and the implant while maintaining axial positioning of the implant with the deployment force delivered by the deployment member, wherein the implant includes a portion having a first length extending distally of distal ends of the deployment member and the retaining member after proximally displacing the retaining member.

5. The method of claim 4, wherein the implant includes a compression member engaged to a distal end thereof, the compression member extending from the implant proximally through the insertion instrument.

6. The method of claim 5, wherein deploying the implant includes the steps of:
c) proximally displacing the distal end of the implant towards the distal ends of the deployment member and the retaining member with the compression member, wherein the proximally displaced portion of the implant includes a second length that is less than the first length.

7. The method of claim 6, further comprising repeating steps a), b) and c) until the proximal end of the implant is displaced distally from the insertion instrument.

8. The method of claim 6, further comprising securing a crimp about the compression member adjacent a proximal end of the implant after proximally displacing the distal end of the implant.

9. The method of claim 1, wherein mounting the implant includes positioning the implant in an elongated passage of a retaining member of the insertion instrument.

10. The method of claim 9, wherein positioning the implant in the elongated passage includes positioning the implant in contact with a deployment member along at least a portion of the length of the implant in the passage.

11. The method of claim 10, wherein deploying the implant includes distally advancing the deployment member in the passage along the retaining member.

12. The method of claim 11, wherein the deployment member is an elongated belt extending along the passage through a distal end opening of the retaining member and proximally from the distal end opening and along the retaining member outside the passage to a mounting member of the insertion instrument adjacent a proximal end of the retaining member.

13. The method of claim 12, wherein the mounting member is a wheel rotatably mounted to the retaining member, and the deployment member extends along at least a portion of the perimeter of the wheel, and deploying the implant includes rotating the wheel.

14. The method of claim 13, wherein the insertion instrument includes a tensioning member outside the passage receiving the deployment member between the mounting member and the distal end of the retaining member.

15. The method of claim 1, wherein deploying the implant includes deploying the implant in a spinal disc space.

16. The method of claim 15, wherein the implant is a spinal disc nucleus replacement device.

17. The method of claim 1, wherein deploying the implant includes deploying the implant in a wall defined by an annulus between adjacent vertebrae.

18. The method of claim 1, wherein the implant includes an elongated body having a plurality of first and second components extending transversely to one another and at a first acute angle relative to a longitudinal axis of the body and deploying the implant includes orienting the plurality of first and second components a second acute angle greater than the first acute angle to axially shorten and radially expand the elongated body of the implant.

19. A surgical system, comprising:
an implant including an elongated, non-rigid body extending between a distal end and a proximal end;
an insertion instrument extending along a longitudinal axis, said implant being mounted along said insertion instrument adjacent a distal end thereof, said insertion instrument including an elongated deployment member extending along and axially moveable relative to an elongated retaining member contactable with said implant, wherein said deployment member includes an engaging portion at a distal end thereof engaging said implant, and further wherein relative axial movement between said deployment member and said retaining member is operable to deploy said implant longitudinally and distally from said distal end of said insertion instrument while said engaging portion engages said implant between said proximal and distal ends of said implant.

20. The system of claim 19, wherein said deployment member is a tubular member and said retaining member is a tubular member received within a passage defined by said deployment member.

21. The system of claim 20, wherein said body of said implant defines a longitudinal bore and in an initial mounting configuration said implant is positioned about said deployment member.

22. The system of claim 21, wherein said deployment member is moveable proximally relative to said retaining member and said implant to position a distal portion of said retaining member within said implant.

23. The system of claim 22, wherein with said distal portion of said retaining member in said implant, said engaging portion engages said implant to maintain a longitudinal positioning of said implant along said deployment member as said retaining member is displaced proximally relative to said deployment member to deploy said implant with a length extending from distal ends of said deployment member and said retaining member.

24. The system of claim 23, wherein said insertion instrument includes a compression member engaged to said distal end of said implant, wherein said compression member is operable to axially compress said deployed length of said implant against said distal ends of said retaining member and said deployment member.

25. The system of claim 24, further comprising a crimp engageable about said compression member in contact with a proximal end of said implant to maintain compression of the said deployed length of said implant.

26. The system of claim 19, wherein said insertion instrument includes a compression member engaged to a distal end of said implant and extending through said retaining member to a proximal end of said insertion instrument.

27. The system of claim 19, wherein said retaining member is received in a passage extending through said deployment member and said engaging portion includes an elongated cannula positioned about said deployment member with said implant between said deployment member and said cannula.

28. The system of claim 19, wherein said retaining member is received in a passage extending through said deployment member and said engaging portion includes a number of distally oriented hooked ends spaced about an outer surface of said deployment member.

29. The system of claim 19, wherein said retaining member includes an elongated tubular member defining a passage and said implant is mounted in said passage.

30. The system of claim 29, wherein said deployment member includes a belt movable along said passage and through a distal end opening of said retaining member, said belt frictionally engaging said implant along at least a portion of a length of said implant between said distal and proximal ends of said implant.

31. The system of claim 30, wherein said insertion instrument includes a wheel mounted to said retaining member and said belt extends at least partially about said wheel, said belt being movable by rotating said wheel.

32. The system of claim 30, further comprising a tensioner between said wheel and a distal end of said retaining member, said belt extending through said tensioner.

33. The system of claim 19, wherein said implant includes an elongated body having a plurality of first and second components extending transversely to one another and at a first acute angle relative to a longitudinal axis of said body, said plurality of first and second components being movable relative to one another to a second acute angle greater than said first acute angle to axially shorten and radially expand said elongated body of the implant when said implant is deployed from said insertion instrument.

34. A surgical system, comprising:
an implant including an elongated, non-rigid body extending between a distal end and a proximal end;
an insertion instrument extending along a longitudinal axis, said insertion instrument including:
an elongated deployment member extending between a distal end and a proximal end;
a retaining member movably received in an elongated passage of said deployment member; and
an engaging portion for engaging said implant, wherein in a first configuration said implant is positioned about said deployment member and a portion of said retaining member extending distally of said deployment member is received within said implant and said retaining member is movable proximally into said deployment member while said engaging portion engages said implant between proximal and distal ends of said implant to maintain a positioning of said implant relative to said deployment member, said implant including an initially deployed configuration when said portion of said retaining member is removed from within said implant.

35. The system of claim 34, wherein said body of said implant defines a longitudinal bore and in an initial mounting configuration said implant is positioned about said deployment member and a distal end of said retaining member is generally aligned with a distal end of said deployment member, said deployment member being axially and proximally movable along said retaining member from said mounting configuration to said first configuration.

36. The system of claim 35, wherein in said initially deployed configuration said implant includes a deployed length extending from distal ends of said deployment member and said retaining member, said deployed length being unsupported by said insertion instrument.

37. The system of claim 36, wherein said insertion instrument includes a compression member engaged to said distal end of said implant, wherein said compression member is operable to axially compress said deployed length of said implant against said distal ends of said deployment member and said retaining member.

38. The system of claim 34, wherein said insertion instrument includes a compression member engaged to a distal end of said implant and extending through said retaining member to a proximal end of said insertion instrument.

39. A surgical system, comprising:
an implant including an elongated, non-rigid body extending between a distal end and a proximal end;
an insertion instrument extending along a longitudinal axis, said insertion instrument including:
an elongated retaining member defining a passage extending along the longitudinal axis between a distal end and a proximal end;
a deployment member movably received in said passage of said retaining member, wherein said implant is positionable in said passage and said deployment member includes a belt movable along said passage and through a distal end opening of said retaining member, said belt frictionally engaging said implant along at least a portion of a length thereof between said distal and proximal ends of said implant.

40. The system of claim 39, wherein said belt forms a continuous loop extending at least in part along said passage and at least in part outside said passage along said retaining member.

41. The system of claim 40, wherein said insertion instrument includes a wheel extending from said retaining member and said belt extends at least partially about said wheel, said wheel being rotatable to move said deployment member along said passage.

42. The system of claim 41, further comprising a tensioner between said mounting wheel and a distal end of said retaining member, said belt extending through said tensioner.

43. A surgical system, comprising:
an insertion instrument extending along a longitudinal axis, said insertion instrument including an elongated deployment member extending along and axially moveable relative to an elongated retaining member; and
an implant including an elongated, non-rigid body extending between a distal end and a proximal end, said implant body being mounted to said insertion instrument adjacent a distal end of said retaining member, said implant being deployable from said distal end of said retaining member upon relative axial movement between said deployment member and said retaining member, wherein said implant includes an elongated body having a plurality of first and second components extending transversely to one another and at a first acute angle relative to a longitudinal axis of said body, said plurality of first and second components being movable relative to one another to a second acute angle greater than said first acute angle to provide an axially shortened and radially expanded form to said elongated body of said implant when deployed from said insertion instrument.

44. The system of claim 43, further comprising:
a compression member extending from said distal end of said implant proximally through said proximal end; and
a crimp engageable about said compression member adjacent said proximal end of said implant to maintain said elongated body in said axially shortened and radially expanded form.
